(19) United States
(12) Patent Application Publication
(10) Pub. No.: US 2024/0091476 A1
(43) Pub. Date: Mar. 21, 2024

(54) SYSTEMS AND METHODS FOR ESTIMATING A SUBJECTIVE COMFORT LEVEL

(71) Applicant: ResMed Sensor Technologies Limited, Dublin (IE)

(72) Inventors: Redmond SHOULDICE, Dublin (IE); Michael WREN, Dublin (IE)

(21) Appl. No.: 18/263,224

(22) PCT Filed: Jan. 27, 2022
(86) PCT No.: PCT/IB2022/050733

§ 371 Date: Jul. 27, 2023

Related U.S. Application Data
(60) Provisional application No. 63/143,381, filed on Jan. 29, 2021.

(51) Int. Cl. A61M 16/00; A61B 5/00; A61B 5/024; A61B 5/08; A61B 5/087; A61B 5/11

(52) U.S. Cl. CPC A61M 16/026; A61B 5/024; A61B 5/0816; A61B 5/087; A61B 5/11; A61B 5/4815; A61B 5/4818

(57) ABSTRACT

A method for predicting a subjective comfort level of a user of a respiratory therapy system is disclosed as follows. Data associated with the user of the respiratory therapy system during a therapy session is received. At least one parameter associated with the user is determined based at least in part on a first portion of the received data. A comfort score is determined based at least in part on the determined at least one parameter. The comfort score is indicative of the subjective comfort level of the user of the respiratory therapy system during at least a portion of the therapy session.

### CROSS-REFERENCE TO RELATED APPLICATIONS

This application claims priority to and the benefit of U.S. Provisional Patent Application No. 63/143,381, filed on Jan. 29, 2021, which is hereby incorporated by reference herein in its entirety.

### TECHNICAL FIELD

The present disclosure relates generally to respiratory therapy systems, and more particularly, to systems and methods for estimating a subjective comfort level of a user of a respiratory therapy system.

### BACKGROUND

Various systems exist for aiding users experiencing sleep apnea and related respiratory disorders. A range of respiratory disorders exist that can impact users. Certain disorders are characterized by particular events (e.g., apneas, hypopneas, hyperpneas, or any combination thereof). Examples of respiratory disorders include periodic limb movement disorder (PLMD), Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and Chest wall disorders.

Respiratory therapy systems can improve the sleep quality of the user. However, if the initial user experience is bad due to poor sleep comfort, the user is more likely to abandon the therapy and sacrifice sleep quality in pursuit of better sleep comfort. Deferment of treatment can lead to deterioration of quality of life due to the user not achieving high quality sleep and/or appropriate therapy of their sleep disordered breathing. Thus, a need exists for systems and methods for estimating a subjective comfort level of a user of a respiratory therapy system. The present disclosure is directed to solving these and other problems.

### SUMMARY

According to some implementations of the present disclosure, a method for estimating a subjective comfort level of a user of a respiratory therapy system is disclosed as follows. Data associated with the user of the respiratory therapy system during a therapy session is received. At least one parameter associated with the user is determined based at least in part on a first portion of the received data. A comfort score is determined based at least in part on the determined at least one parameter. The comfort score is indicative of the subjective comfort level of the user of the respiratory therapy system during at least a portion of the therapy session.

According to some implementations of the present disclosure, a system includes a control system and a memory. The control system includes one or more processors. The memory has stored thereon machine readable instructions. The control system is coupled to the memory. Any one of the methods disclosed herein is implemented when the machine executable instructions in the memory are executed by at least one of the one or more processors of the control system.

According to some implementations of the present disclosure, a system for estimating a subjective comfort level of a user of a respiratory therapy system is disclosed as follows. The system includes a control system configured to implement any one of the methods disclosed herein.

According to some implementations of the present disclosure, a computer program product comprising instructions which, when executed by a computer, cause the computer to carry out any one of the methods disclosed herein. In some implementations, the computer program product is a non-transitory computer readable medium.

According to some implementations of the present disclosure, a system includes a control system and a memory. The control system includes one or more processors. The memory stores machine readable instructions. The control system is coupled to the memory, and is configured to implement a method for estimating a subjective comfort level of a user of a respiratory therapy system. The method includes receiving data associated with the user of the respiratory therapy system during a therapy session. The method further includes determining at least one parameter associated with the user based at least in part on a first portion of the received data. The method further includes determining a comfort score based at least in part on the determined at least one parameter, the comfort score being indicative of the subjective comfort level of the user of the respiratory therapy system during at least a portion of the therapy session.

The foregoing and additional aspects and implementations of the present disclosure will be apparent to those of ordinary skill in the art in view of the detailed description of various embodiments and/or implementations, which is made with reference to the drawings, a brief description of which is provided next.

### BRIEF DESCRIPTION OF THE DRAWINGS

The foregoing and other advantages of the present disclosure will become apparent upon reading the following detailed description and upon reference to the drawings.

FIG. 1 is a functional block diagram of a system for estimating a subjective comfort level of a user of a respiratory therapy system, according to some implementations of the present disclosure.

FIG. 2 is a perspective view of at least a portion of the system of FIG. 1, a user wearing a full face mask, and a bed partner, according to some implementations of the present disclosure.

FIG. 3 illustrates a flow diagram for a method for estimating a subjective comfort level of a user of a respiratory therapy system, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration.

Various systems exist for aiding users experiencing sleep apnea and related respiratory disorders. A range of respiratory disorders exist that can impact users. Examples of respiratory disorders include periodic limb movement disorder (PLMD), Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and Chest wall disorders. These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea. The AHI calculated based on apnea and/or hypopnea events experienced by the user during the sleep session and while on respiratory therapy is known as “residual” AHI.

In order to mitigate some of these sleep-related and/or respiratory disorders, a user can be prescribed usage of a respiratory device or system. For example, a continuous positive airway pressure (CPAP) machine can be used to increase air pressure in the throat of the respiratory device user and to prevent the airway from closing and/or narrowing during sleep. Therefore, a goal of the therapy is to reduce the AHI, ideally to normal (or otherwise acceptable) levels, and improve the sleep quality of the user.

While these respiratory devices or systems can improve the sleep quality of the user, the comfort of the user is sometimes compromised. For example, while attempting to sleep a user may feel discomfort due to air being forced into their airways. Other discomforts might be related to sounds of the system, sweating or stickiness caused by or associated with a face mask, or discomfort and perceived claustrophobia associated with wearing a face mask. After a sleep session, a user can experience belching, stomach bloating, stomach distension and agonizing gas pains due to aerophagia. Aerophagia occurs when air enters the esophagus and goes into the stomach instead of the air entering the airways and to the lungs as intended. Other discomforts after a sleep session can include dryness of the nose, throat, or eyes as a result of leaks causing high air flow or poor humidification of the therapeutic air.

While patient discomfort can be identified based on subjective feedback from the user, objective measures can also be used to identify discomfort, alone or in combination with the subjective feedback. For example, detection of eye blink and movements of the facial muscles due the physiological reaction to jetting of air from a poor mask seal, which may be drying or otherwise irritating the eyes can be used to infer a discomfort due to the unintentional leak.

The discomfort experienced by a person who is prescribed use of a respiratory therapy system is often more pronounced when the user is first adopting a sleep therapy using the system. If the initial user experience is bad due to poor therapy comfort, the user is more likely to abandon the therapy, or sacrifice therapy and/or sleep quality in pursuit of better therapy and/or sleep comfort. Deferment of treatment can lead to deterioration of quality of life due to the user not achieving high quality sleep and/or appropriate therapy of their sleep disordered breathing.

In some instances, the user's experience is not fully reflected in the current therapy score. For example, the myAir™ score (a proprietary therapy score by ResMed) is calculated based on usage hours, user interface seal/leak, events per hour, and mask on/off. In addition, the experience of comfort is rather subjective to the user. A female user can have a lower AHI, but may feel more personal discomfort than a male user. In particular, male users may have fewer events in non-REM, but can have many during REM. A female user with relatively low AHI compared to a male user can have similar symptoms to the male user, but with a much higher AHI if the female user's REM sleep is particularly heavily disturbed by apnea events. As described further herein, a comfort score can feed into the determination of a therapy and/or sleep score by, for example, being a further component of the therapy and/or sleep score, or weighting or adjusting one or more components of the therapy and/or sleep score based on the comfort score, or a combination thereof.

While comfort level is a subjective feeling, it can have objective signatures. The present disclosure is directed to systems and methods for estimating a subjective comfort level of a user (such as outputting a comfort score), based at least in part on those objective signatures. If a user knows that their comfort score is low, which may not be normal or may be below a threshold, they may be motivated, and guided, to improve it.

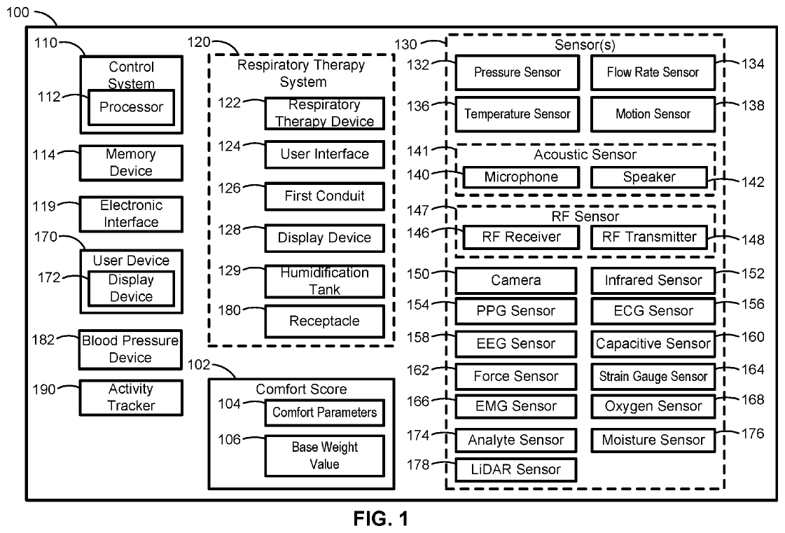

Referring to FIG. 1, a functional block diagram is illustrated, of a system 100 for estimating a subjective comfort level of a user of a respiratory therapy system. The system 100 includes a comfort score module 102, a control system 110, a memory device 114, an electronic interface 119, one or more sensors 130, and one or more user devices 170. In some implementations, the system 100 further optionally includes a respiratory therapy system 120, a blood pressure device 182, an activity tracker 190, or any combination thereof.

The comfort score module 102 determines a comfort score for a user based at least on comfort parameters 104 (e.g., calculated and/or derived from comfort-related parameters) and, optionally, base weight values 106. The comfort score is indicative of the subjective comfort level of a user of the respiratory therapy system. The comfort parameters 104 are determined based on data such as can be collected by, for example, the sensors 130 and/or inputted by the user. The base weight values 106 are modifiers applied to the comfort parameters depending on the importance of a specific parameter, the impact of that specific parameter on the user's comfort level (or relative impact compared to one or more other parameters), or the correlation of that specific parameter with the user's comfort level (or relative correlation compared to one or more other parameters). That is, the comfort score is a function of both the comfort parameters 104 and the base weight values 106. The comfort score can be any useful representation or value, such as a number, a word, a string of text, a letter a symbol or a string of machine-readable code.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, the activity tracker 190, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory device 122, within a housing of the user device 170, the activity tracker 190, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with the user, which can be implemented as comfort parameters 104 for determination of the comfort score. The user profile can include, for example, demographic information associated with the user, biometric information associated with the user, medical information associated with the user, self-reported user feedback, sleep parameters associated with the user (e.g., sleep-related parameters recorded from one or more sleep sessions), or any combination thereof. The demographic information can include, for example, information indicative of an age of the user, a gender of the user, a race of the user, an ethnicity of the user, a geographic location of the user, a travel history of the user, a relationship status, a status of whether the user has one or more pets, a status of whether the user has a family, a family history of health conditions, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) test result or score and/or a Pittsburgh Sleep Quality Index (PSQI) score or value. The medical information data can include results from one or more of a polysomnography (PSG) test, a CPAP titration, or a home sleep test (HST), respiratory therapy system settings from one or more sleep sessions, sleep related respiratory events from one or more sleep sessions, or any combination thereof. The self-reported user feedback can include information indicative of a self-reported subjective therapy score (e.g., poor, average, excellent), a self-reported subjective stress level of the user, a self-reported subjective fatigue level of the user, a self-reported subjective health status of the user, a recent life event experienced by the user, or any combination thereof. The user profile information can be updated at any time, such as daily (e.g. between sleep sessions), weekly, monthly or yearly. In some implementations, the memory device 114 stores media content that can be displayed on the display device 128 and/or the display device 172.

The electronic interface 119 is configured to receive data (e.g., physiological data, flow rate data, pressure data, motion data, acoustic data, etc.) from the one or more sensors 130 such that the data can be stored in the memory device 114 and/or analyzed by the processor 112 of the control system 110. The received data, such as physiological data, flow rate data, pressure data, motion data, acoustic data, etc., may be used to determine and/or calculate comfort parameters 104 for determination of the comfort score. The electronic interface 119 can communicate with the one or more sensors 130 using a wired connection or a wireless connection (e.g., using an RF communication protocol, a Wi-Fi communication protocol, a Bluetooth communication protocol, an IR communication protocol, over a cellular network, over any other optical communication protocol, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

The respiratory therapy system 120 can include a respiratory pressure therapy (RPT) device 122 (referred to herein as respiratory device 122 or respiratory therapy device), a user interface 124, a conduit 126 (also referred to as a tube or an air circuit), a display device 128, a humidification tank 129, a receptacle 180 or any combination thereof. In some implementations, the control system 110, the memory device 114, the display device 128, one or more of the sensors 130, and the humidification tank 129 are part of the respiratory device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory device 122 is generally used to generate pressurized air that is delivered to a user (e.g., using one or more motors that drive one or more compressors). In some implementations, the respiratory device 122 generates continuous constant air pressure that is delivered to the user. In other implementations, the respiratory device 122 generates two or more predetermined pressures (e.g., a first predetermined air pressure and a second predetermined air pressure). In still other implementations, the respiratory device 122 is configured to generate a variety of different air pressures within a predetermined range. For example, the respiratory device 122 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory device 122 to the user's airway to aid in preventing the airway from narrowing and/or collapsing during sleep. This may also increase the user's oxygen intake during sleep. Generally, the user interface 124 engages the user's face such that the pressurized air is delivered to the user's airway via the user's mouth, the user's nose, or both the user's mouth and nose. Together, the respiratory device 122, the user interface 124, and the conduit 126 form an air pathway fluidly coupled with an airway of the user. The pressurized air also increases the user's oxygen intake during sleep.

Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cmH₂O relative to ambient pressure. For other forms of therapy, such as the delivery of oxygen, the user interface may not include a seal sufficient to facilitate delivery to the airways of a supply of gas at a positive pressure of about 10 cmH₂O.

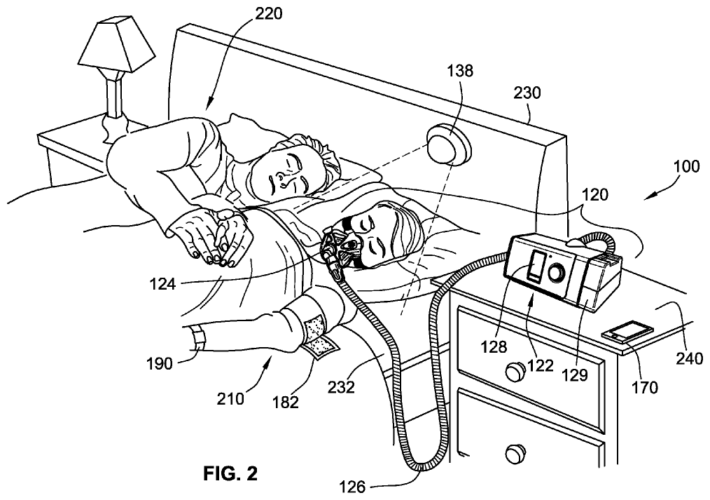

As shown in FIG. 2, in some implementations, the user interface 124 is or includes a facial mask (e.g., a full face mask) that covers the nose and mouth of the user. Alternatively, in some implementations, the user interface 124 is a nasal mask that provides air to the nose of the user or a nasal pillow mask that delivers air directly to the nostrils of the user. The user interface 124 can include a plurality of straps (e.g., including hook and loop fasteners) for positioning and/or stabilizing the interface on a portion of the user (e.g., the face) and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210. In other implementations, the user interface 124 includes a mouthpiece (e.g., a night guard mouthpiece molded to conform to the user's teeth, a mandibular repositioning device, etc.).

The conduit 126 (also referred to as an air circuit or tube) allows the flow of air between two components of the respiratory therapy system 120, such as the respiratory device 122 and the user interface 124. In some implementations, there can be separate limbs of the conduit for inhalation and exhalation. In other implementations, a single limb conduit is used for both inhalation and exhalation.

One or more of the respiratory device 122, the user interface 124, the conduit 126, the display device 128, and the humidification tank 129 can contain one or more sensors (e.g., a pressure sensor, a flow rate sensor, a humidity sensor, a temperature sensor, or more generally any of the other sensors 130 described herein). These one or more sensors can be used, for example, to measure the air pressure and/or flow rate of pressurized air supplied by the respiratory device 122.

The display device 128 is generally used to display image(s) including still images, video images, or both and/or information regarding the respiratory device 122. For example, the display device 128 can provide information regarding the status of the respiratory device 122 (e.g., whether the respiratory device 122 is on/off, the pressure of the air being delivered by the respiratory device 122, the temperature of the air being delivered by the respiratory device 122, etc.) and/or other information (e.g., a sleep score and/or a therapy score (also referred to as a myAir™ score, such as described in WO 2016/061629 and U.S. Patent Pub. No. 2017/0311879, each of which is hereby incorporated by reference herein in its entirety), the current date/time, personal information for the user 210, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory device 122.

The humidification tank 129 is coupled to or integrated in the respiratory device 122. The humidification tank 129 includes a reservoir of water that can be used to humidify the pressurized air delivered from the respiratory device 122. The respiratory device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user. The humidification tank 129 can be fluidly coupled to a water vapor inlet of the air pathway and deliver water vapor into the air pathway via the water vapor inlet, or can be formed in-line with the air pathway as part of the air pathway itself. In other implementations, the respiratory device 122 or the conduit 126 can include a waterless humidifier. The waterless humidifier can incorporate sensors that interface with other sensor positioned elsewhere in system 100.

In some implementations, the system 100 can be used to deliver at least a portion of a substance from a receptacle 180 to the air pathway the user based at least in part on the physiological data, the sleep-related parameters, other data or information, or any combination thereof. Generally, modifying the delivery of the portion of the substance into the air pathway can include (i) initiating the delivery of the substance into the air pathway, (ii) ending the delivery of the portion of the substance into the air pathway, (iii) modifying an amount of the substance delivered into the air pathway, (iv) modifying a temporal characteristic of the delivery of the portion of the substance into the air pathway, (v) modifying a quantitative characteristic of the delivery of the portion of the substance into the air pathway, (vi) modifying any parameter associated with the delivery of the substance into the air pathway, or (vii) any combination of (i)-(vi).

Modifying the temporal characteristic of the delivery of the portion of the substance into the air pathway can include changing the rate at which the substance is delivered, starting and/or finishing at different times, continuing for different time periods, changing the time distribution or characteristics of the delivery, changing the amount distribution independently of the time distribution, etc. The independent time and amount variation ensures that, apart from varying the frequency of the release of the substance, one can vary the amount of substance released each time. In this manner, a number of different combination of release frequencies and release amounts (e.g., higher frequency but lower release amount, higher frequency and higher amount, lower frequency and higher amount, lower frequency and lower amount, etc.) can be achieved. Other modifications to the delivery of the portion of the substance into the air pathway can also be utilized.

The respiratory therapy system 120 can be used, for example, as a ventilator or a positive airway pressure (PAP) system such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory therapy system 120 and a bed partner 220 are located in a bed 230 and are laying on a mattress 232. A motion sensor 138, a blood pressure device 182, and an activity tracker 190 are shown, although any one or more sensors 130 can be used to generate or monitor the comfort parameters 104 during a therapy, sleeping, and/or resting session of the user 210.

The user interface 124 is a facial mask (e.g., a full face mask) that covers the nose and mouth of the user 210. Alternatively, the user interface 124 can be a nasal mask that provides air to the nose of the user 210 or a nasal pillow mask that delivers air directly to the nostrils of the user 210. The user interface 124 can include a plurality of straps (e.g., including hook and loop fasteners) for positioning and/or stabilizing the interface on a portion of the user 210 (e.g., the face) and a conformal cushion (e.g., silicone, plastic, foam, etc.) that aids in providing an air-tight seal between the user interface 124 and the user 210. The user interface 124 can also include one or more vents for permitting the escape of carbon dioxide and other gases exhaled by the user 210. In other implementations, the user interface 124 is a mouthpiece (e.g., a night guard mouthpiece molded to conform to the user's teeth, a mandibular repositioning device, etc.) for directing pressurized air into the mouth of the user 210.

The user interface 124 is fluidly coupled and/or connected to the respiratory device 122 via the conduit 126. In turn, the respiratory device 122 delivers pressurized air to the user 210 via the conduit 126 and the user interface 124 to increase the air pressure in the throat of the user 210 to aid in preventing the airway from closing and/or narrowing during sleep. The respiratory device 122 can be positioned on a nightstand 240 that is directly adjacent to the bed 230 as shown in FIG. 2, or more generally, on any surface or structure that is generally adjacent to the bed 230 and/or the user 210.

Generally, a user who is prescribed usage of the respiratory therapy system 120 will tend to experience higher quality sleep and less fatigue during the day after using the respiratory therapy system 120 during the sleep compared to not using the respiratory therapy system 120 (especially when the user suffers from sleep apnea or other sleep related disorders). For example, the user 210 may suffer from obstructive sleep apnea and rely on the user interface 124 (e.g., a full face mask) to deliver pressurized air from the respiratory device 122 via conduit 126. The respiratory device 122 can be a continuous positive airway pressure (CPAP) machine used to increase air pressure in the throat of the user 210 to prevent the airway from closing and/or narrowing during sleep. For someone with sleep apnea, their airway can narrow or collapse during sleep, reducing oxygen intake, and forcing them to wake up and/or otherwise disrupt their sleep. The CPAP machine prevents the airway from narrowing or collapsing, thus minimizing the occurrences where she wakes up or is otherwise disturbed due to reduction in oxygen intake. While the respiratory device 122 strives to maintain a medically prescribed air pressure or pressures during sleep, the user can experience sleep discomfort due to the therapy.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a Light Detection and Ranging (LiDAR) sensor 178, an electrodermal sensor, an accelerometer, an electrooculography (EOG) sensor, a light sensor, a humidity sensor, an air quality sensor, or any combination thereof. Generally, each of the one or more sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the Light Detection and Ranging (LiDAR) sensor 178 more generally, the one or more sensors 130 can include any combination and any number of each of the sensors described and/or shown herein.

Data from room environment sensors can also be incorporated into the comfort score, such as temperature throughout a sleep session (e.g., too warm, too cold), humidity (e.g., too high, too low), pollution levels (e.g., an amount and/or concentration of CO₂ and/or particulates being under or over a threshold), light levels (e.g., too bright, not using blackout blinds, too much blue light before falling asleep), and sound levels (e.g., above a threshold, types of sources, linked to interruptions in sleep, snoring of a partner). These can be captured by sensors on a respiratory therapy device, by a smartphone (e.g., connected via Bluetooth or internet), or separate sensors (such as connected to a home automation system). An air quality sensor can also detect other types of pollution in the room than cause allergies, such as from pets, dust mites, and so forth—and where the room could benefit from air filtration in order to increase use comfort of the user.

Data from the user's health (physical and/or mental) condition can also be incorporated. For example, certain detected changes in comfort may not solely be related to changes in therapy equipment (or suitable of selected equipment) or the room/bed environment. Variation in comfort can also relate to health (such as a change due to the onset or offset of illness such as respiratory issue, and/or due to a change in an underlying condition such as a co-morbid chronic condition).

For example, PPG data from the PPG sensor 154 (such as on the mask, headgear, as a patch, as a watch, as a finger probe, a ring, or in/on the ear, etc.) could be used to estimate heart rate, pulse rate (PR), blood pressure, SpO₂, and changes in peripheral arterial tone. The blood oxygenation level could be referenced to the PAP therapy to confirm that no unexpected drops are seen—and also if/when the therapy is off (such as mask removed) to monitor any residual respiratory (e.g. apnea) events. These PPG data can be used to estimate likely daytime headache, and/or suggest a change to PAP therapy, such as further treating flow limitations in addition to pure obstructive events. These PPG data can also be used to check for an inflammation response. Headaches could also be due to a pressure setting that is too high, and might benefit from a reduced pressure, or change to an EPR setting.

A PAT (peripheral arterial tone) sensing device may make use of a fingertip mounted PPG probe. The PPG probe operates with an optical technology that detects blood volume changes in the tissue's microvascular bed. As noted above, PPG measurements are used to derive the arterial blood oxygen saturation (SpO₂), pulse rate (PR), and changes in peripheral arterial tone, which are then used to detect respiratory events. Peripheral arterial tone refers to the tone of the peripheral arterial smooth muscle tissue. When the muscle tone of peripheral arteries increases, the arteries' diameter decreases, resulting in a reduction of perfusion and thus a decrease in pulsatile blood volume in the peripheral tissue. The decrease in pulsatile blood volume in the peripheral tissue is picked up as a drop in the PPG signal swing between systole and diastole. The PAT signal may be derived from the PPG signal from the PPG sensor, such as by the method described in WO 2021/260190, the disclosure of which is incorporated by reference herein in its entirety. The PPG-derived signal, which may be derived by trending such pulsatile blood volume reductions, is referred to as the PAT signal.

A PAT sensing device analyzes the concurrence of drops in SpO₂, surges in pulse rate, and increases in peripheral arterial tone. As airflow is reduced, such as during a respiratory (e.g., apnea) event, the oxygen supply to the lungs decreases from baseline, resulting in distinct SpO₂ drops. Near the end of the respiratory event, the sympathetic nervous system activity spikes to arouse the patient, resulting in resumption of ventilation, a surge in pulse rate and a release of norepinephrine in the blood stream. Consequently, norepinephrine binds to alpha-adrenergic receptors innervating the arterial smooth muscle tissue in the finger, causing sudden vasoconstrictions, or synchronously increases in PAT, picked up by the PPG sensor. Such a PPG sensor may be implemented for detection of respiratory events, such as by detecting signal characteristics indicative of apnea and/or hypopnea events within a peripheral arterial tonometry signal that can be derived from the sensor's PPG signal. The PPG signal may also be processed to extract respiratory effort-related information from which different types of respiratory events may be determined.

A photo using a camera and/or a mapping of the face with a RADAR or LiDAR can be sued to check for mask tightness (e.g., over tightening or under tightening), and/or for any marks on the face or head (such as from chafing or skin irritation—showing a change in skin color where the patient interface was from expected normal). Skin irritation may lead to the system to suggest a different type of mask (including a similar form factor, but different shape such as to better seal with comfort to the face), a different size of mask, or a different type of cushioning material. The camera 150 can also be used to check for any facial hair, and possible interaction with the mask seal.

An analysis of sleep quality based on processing of sensors can be used to check for insomnia (including due to hyperarousal, as determined via a person's temperature and/or heart rate elevation). The system described herein can link detected possible discomfort factors to acute insomnia, such as the onset of insomnia due to a difficulty in falling asleep, staying asleep, or waking up earlier than expected or desired.

As described herein, the system 100 generally can be used to generate data (e.g., physiological data, flow rate data, pressure data, motion data, acoustic data, etc.) associated with a user (e.g., a user of the respiratory therapy system 120 shown in FIG. 2) during a sleep session. The generated data can be analyzed to generate one or more sleep-related parameters, which can include any parameter, measurement, etc. related to the user during the sleep session. The one or more sleep-related parameters that can be determined for the user 210 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory device 122, a heart rate, a heart rate variability, movement of the user 210, temperature, EEG activity, EMG activity, arousal, snoring, choking, coughing, whistling, wheezing, or any combination thereof.

The one or more sensors 130 can be used to generate, for example, physiological data, flow rate data, pressure data, motion data, acoustic data, etc. In some implementations, the data generated by one or more of the sensors 130 can be used by the control system 110 to determine the duration of sleep and sleep quality of user 210, which is a comfort parameter 104. For example, a sleep-wake signal associated with the user 210 during the sleep session and one or more sleep-related parameters. The sleep-wake signal can be indicative of one or more sleep states, including sleep, wakefulness, relaxed wakefulness, micro-awakenings, or distinct sleep stages such as a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more of the sensors, such as sensors 130, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

The sleep-wake signal can also be timestamped to determine a time that the user enters the bed, a time that the user exits the bed, a time that the user attempts to fall asleep, etc. The sleep-wake signal can be measured by the one or more sensors 130 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory device 122, or any combination thereof during the sleep session.

The event(s) can include snoring, apneas, central apneas, obstructive apneas, mixed apneas, hypopneas, a mouth leak, a mask leak (e.g., from the user interface 124), a restless leg, a sleeping disorder, choking, an increased heart rate, a heart rate variation, labored breathing, an asthma attack, an epileptic episode, a seizure, a fever, a cough, a sneeze, a snore, a gasp, the presence of an illness such as the common cold or the flu, or any combination thereof. In some implementations, mouth leak can include continuous mouth leak, or valve-like mouth leak (i.e. varying over the breath duration) where the lips of a user, typically using a nasal/nasal pillows mask, pop open on expiration. Mouth leak can lead to dryness of the mouth, bad breath, and is sometimes colloquially referred to as “sandpaper mouth.” The event(s) can also include carbon dioxide (CO₂) build up in the user interface 124 or other part of the respiratory therapy system 120, which can be uncomfortable for a user. Carbon dioxide (CO₂) levels may be detected by, for example, one or more CO₂ sensors in the user interface 124 and/or other part of the respiratory therapy system 120.

The one or more sleep-related parameters that can be determined for the user during the sleep session based on the sleep-wake signal include, for example, sleep quality metrics such as a total time in bed, a total sleep time, a sleep onset latency, a wake-after-sleep-onset parameter, a sleep efficiency, a fragmentation index, or any combination thereof.

The data generated by the one or more sensors 130 (e.g., physiological data, flow rate data, pressure data, motion data, acoustic data, etc.) can also be used to determine a respiration signal associated with a user during a sleep session. The respiration signal is generally indicative of respiration or breathing of the user during the sleep session. The respiration signal can be indicative of, for example, a respiration rate, a respiration rate variability, an inspiration amplitude, an expiration amplitude, an inspiration-expiration ratio, a number of events per hour, a pattern of events, pressure settings of the respiratory device 122, or any combination thereof. The event(s) can include snoring, apneas, central apneas, obstructive apneas, mixed apneas, hypopneas, a mouth leak, a mask leak (e.g., from the user interface 124), a restless leg, a sleeping disorder, choking, an increased heart rate, labored breathing, an asthma attack, an epileptic episode, a seizure, or any combination thereof. As discussed herein, one or more sleep- and/or respiration-related parameters can be, or can be indicative of, one or more comfort-related parameters from which a comfort score can be derived.

Generally, the sleep session includes any point in time after the user 210 has laid or sat down in the bed 230 (or another area or object on which they intend to sleep), and/or has turned on the respiratory device 122 and/or donned the user interface 124. The sleep session can thus include time periods (i) when the user 210 is using the CPAP system but before the user 210 attempts to fall asleep (for example when the user 210 lays in the bed 230 reading a book); (ii) when the user 210 begins trying to fall asleep but is still awake; (iii) when the user 210 is in a light sleep (also referred to as stage 1 and stage 2 of non-rapid eye movement (NREM) sleep); (iv) when the user 210 is in a deep sleep (also referred to as slow-wave sleep, SWS, or stage 3 of NREM sleep); (v) when the user 210 is in rapid eye movement (REM) sleep; (vi) when the user 210 is periodically awake between light sleep, deep sleep, or REM sleep; or (vii) when the user 210 wakes up and does not fall back asleep.

The sleep session is generally defined as ending once the user 210 removes the user interface 124, turns off the respiratory device 122, and/or gets out of bed 230. In some implementations, the sleep session can include additional periods of time, or can be limited to only some of the above-disclosed time periods. For example, the sleep session can be defined to encompass a period of time beginning when the respiratory device 122 begins supplying the pressurized air to the airway or the user 210, ending when the respiratory device 122 stops supplying the pressurized air to the airway of the user 210, and including some or all of the time points in between, when the user 210 is asleep or awake.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory device 122. the user interface 124, or the conduit 126. The pressure sensor 132 can be used to determine an air pressure in the respiratory device 122, an air pressure in the conduit 126, an air pressure in the user interface 124, or any combination thereof. The pressure sensor 132 can be, for example, a capacitive sensor, an electromagnetic sensor, an inductive sensor, a resistive sensor, a piezoelectric sensor, a strain-gauge sensor, an optical sensor, a potentiometric sensor, or any combination thereof. In one example, the pressure sensor 132 can be used to determine a blood pressure of a user.

The flow rate sensor 134 outputs flow rate data that can be stored in the memory device 114 and/or analyzed by the processor 112 of the control system 110. In some implementations, the flow rate sensor 134 is used to determine an air flow rate from the respiratory device 122, an air flow rate through the conduit 126, an air flow rate through the user interface 124, or any combination thereof. In such implementations, the flow rate sensor 134 can be coupled to or integrated in the respiratory device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The flow rate sensor 134 can be used to generate flow rate data associated with the user 210 (FIG. 2) of the respiratory device 122 during the sleep session. Examples of flow rate sensors (such as, for example, the flow rate sensor 134) are described in WO 2012/012835 and U.S. Pat. No. 10,328,219, each of which is hereby incorporated by reference herein in its entirety. In some implementations, the flow rate sensor 134 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user.

The temperature sensor 136 outputs temperature data that can be stored in the memory device 114 and/or analyzed by the processor 112 of the control system 110. In some implementations, the temperature sensor 136 generates temperature data indicative of a core body temperature of the user 210 (FIG. 2), a skin temperature of the user 210, a temperature of the air flowing from the respiratory device 122 and/or through the conduit 126, a temperature of the air in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user 210 during the sleep session, and/or detect movement of any of the components of the respiratory therapy system 120, such as the respiratory device 122, the user interface 124, or the conduit 126. The motion sensor 138 can include one or more inertial sensors, such as accelerometers, gyroscopes, and magnetometers. In some implementations, the motion sensor 138 alternatively or additionally generates one or more signals representing bodily movement of the user, from which may be obtained a signal representing a sleep state or sleep stage of the user; for example, via a respiratory movement of the user. In some implementations, the motion data from the motion sensor 138 can be used in conjunction with additional data from another sensor 130 to determine the sleep state or sleep stage of the user. In some implementations, the motion data can be used to determine a location, a body position, and/or a change in body position of the user.

The microphone 140 outputs sound data that can be stored in the memory device 114 and/or analyzed by the processor 112 of the control system 110. The microphone 140 can be used to record sound(s) during a sleep session (e.g., sounds from the user 210) to determine (e.g., using the control system 110) one or more sleep related parameters, which may include one or more events (e.g., respiratory events), as described in further detail herein. The microphone 140 can be coupled to or integrated in the respiratory device 122, the user interface 124, the conduit 126, or the user device 170. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones.

The speaker 142 outputs sound waves. In one or more implementations, the sound waves can be audible to a user of the system 100 (e.g., the user 210 of FIG. 2) or inaudible to the user of the system (e.g., ultrasonic sound waves). The speaker 142 can be used, for example, as an alarm clock or to play an alert or message to the user 210 (e.g., in response to an identified body position and/or a change in body position). In some implementations, the speaker 142 can be used to communicate the audio data generated by the microphone 140 to the user. The speaker 142 can be coupled to or integrated in the respiratory device 122, the user interface 124, the conduit 126, or the user device 170.

The microphone 140 and the speaker 142 can be used as separate devices. In some implementations, the microphone 140 and the speaker 142 can be combined into an acoustic sensor 141 (e.g. a SONAR sensor), as described in, for example, WO 2018/050913 and WO 2020/104465, each of which is hereby incorporated by reference herein in its entirety. In such implementations, the speaker 142 generates or emits sound waves at a predetermined interval and/or frequency and the microphone 140 detects the reflections of the emitted sound waves from the speaker 142. In one or more implementations, the sound waves generated or emitted by the speaker 142 can have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters (e.g., an identified body position and/or a change in body position) described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory device 122, or any combination thereof. In this context, a sonar sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO2018/050913 and WO 2020/104465 mentioned above.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location and/or a body position of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g. a RADAR sensor). In some such implementations, the RF sensor 147 includes a control circuit. The specific format of the RF communication could be Wi-Fi, Bluetooth, or etc.

In some implementations, the RF sensor 147 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 147. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or any combination thereof) that can be stored in the memory device 114. The image data from the camera 150 can be used by the control system 110 to determine one or more of the sleep-related parameters described herein. The image data from the camera 150 can be used by the control system 110 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 150 can be used to identify a location and/or a body position of the user, to determine chest movement of the user 210, to determine air flow of the mouth and/or nose of the user 210, to determine a time when the user 210 enters the bed 230, and to determine a time when the user 210 exits the bed 230. The camera 150 can also be used to track eye movements, pupil dilation (if one or both of the user 210's eyes are open), blink rate, or any changes during REM sleep.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate pattern, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state or sleep stage of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the user 210's breath. In some implementations, the analyte sensor 174 is positioned near the user 210's mouth to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the user 210's nose to detect analytes in breath exhaled through the user's nose. In still other implementations, the analyte sensor 174 can be positioned near the user 210's mouth when the user interface 124 is a nasal mask or a nasal pillow mask. In some implementations, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the user 210's mouth or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory device 122, etc.). Thus, in some implementations, the moisture sensor 176 can be positioned in the user interface 124 or in the conduit 126 to monitor the humidity of the pressurized air from the respiratory device 122. In other implementations, the moisture sensor 176 is placed near any area where moisture levels need to be monitored. The moisture sensor 176 can also be used to monitor the humidity of the ambient environment surrounding the user 210, for example, the air inside the user 210's bedroom. The moisture sensor 176 can also be used to track the user 210's biometric response to environmental changes.

One or more Light Detection and Ranging (LiDAR) sensors 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 178 can measure and map an area extending 5 meters or more away from the sensor. The LiDAR data can be fused with point cloud data estimated by an electromagnetic RADAR sensor, for example. The LiDAR sensor(s) 178 may also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 130 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, an orientation sensor, a rain sensor, a soil moisture sensor, a water flow sensor, an alcohol sensor, or any combination thereof.

While shown separately in FIG. 1, any combination of the one or more sensors 130 can be integrated in and/or coupled to any one or more of the components of the system 100, including the respiratory device 122, the user interface 124, the conduit 126, the humidification tank 129, the control system 110, the user device 170, or any combination thereof. For example, the acoustic sensor 141 and/or the RF sensor 147 can be integrated in and/or coupled to the user device 170. In such implementations, the user device 170 can be considered a secondary device that generates additional or secondary data for use by the system 100 (e.g., the control system 110) according to some aspects of the present disclosure. In some implementations, at least one of the one or more sensors 130 is not physically and/or communicatively coupled to the respiratory device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The data from the one or more sensors 130 can be analyzed to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, a sleep stage, an apnea-hypopnea index (AHI), or any combination thereof. The one or more events can include snoring, apneas, central apneas, obstructive apneas, mixed apneas, hypopneas, an intentional mask leak, an unintentional mask leak, a mouth leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Non-physiological parameters can also include operational parameters of the respiratory therapy system, including flow rate, pressure, humidity of the pressurized air, speed of motor, etc. Other types of physiological and non-physiological parameters can also be determined, either from the data from the one or more sensors 130, or from other types of data.

The user device 170 (FIG. 1) includes a display device 172. The user device 170 can be, for example, a mobile device such as a smart phone, a tablet, a gaming console, a smart watch, a laptop, or the like. Alternatively, the user device 170 can be an external sensing system, a television (e.g., a smart television) or another smart home device (e.g., a smart speaker(s) such as Google Home™, Google Nest™, Amazon Echo™, Amazon Echo Show™, Alexa™-enabled devices, etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

The blood pressure device 182 is generally used to aid in generating physiological data for determining one or more blood pressure measurements associated with a user. The blood pressure device 182 can include at least one of the one or more sensors 130 to measure, for example, a systolic blood pressure component and/or a diastolic blood pressure component.

In some implementations, the blood pressure device 182 is a sphygmomanometer including an inflatable cuff that can be worn by a user and a pressure sensor (e.g., the pressure sensor 132 described herein). For example, as shown in the example of FIG. 2, the blood pressure device 182 can be worn on an upper arm of the user 210. In such implementations where the blood pressure device 182 is a sphygmomanometer, the blood pressure device 182 also includes a pump (e.g., a manually operated bulb) for inflating the cuff. In some implementations, the blood pressure device 182 is coupled to the respiratory device 122 of the respiratory therapy system 120, which in turn delivers pressurized air to inflate the cuff. More generally, the blood pressure device 182 can be communicatively coupled with, and/or physically integrated in (e.g., within a housing), the control system 110, the memory 114, the respiratory therapy system 120, the user device 170, and/or the activity tracker 190.

The activity tracker 190 is generally used to aid in generating physiological data for determining an activity measurement associated with the user. The activity measurement can include, for example, a number of steps, a distance traveled, a number of steps climbed, a duration of physical activity, a type of physical activity, an intensity of physical activity, time spent standing, a respiration rate, an average respiration rate, a resting respiration rate, a maximum respiration rate, a respiration rate variability, a heart rate, an average heart rate, a resting heart rate, a maximum heart rate, a heart rate variability, a number of calories burned, blood oxygen saturation level (SpO₂), electrodermal activity (also known as skin conductance or galvanic skin response), a position of the user, a posture of the user, or any combination thereof. The activity tracker 190 includes one or more of the sensors 130 described herein, such as, for example, the motion sensor 138 (e.g., one or more accelerometers and/or gyroscopes), the PPG sensor 154, and/or the ECG sensor 156.

In some implementations, the activity tracker 190 is a wearable device that can be worn by the user, such as a smartwatch, a wristband, a ring, or a patch. For example, referring to FIG. 2, the activity tracker 190 is worn on a wrist of the user 210. The activity tracker 190 can also be coupled to or integrated a garment or clothing that is worn by the user. Alternatively still, the activity tracker 190 can also be coupled to or integrated in (e.g., within the same housing) the user device 170. More generally, the activity tracker 190 can be communicatively coupled with, or physically integrated in (e.g., within a housing), the control system 110, the memory 114, the respiratory therapy system 120, and/or the user device 170, and/or the blood pressure device 182.

While the control system 110 and the memory device 114 are described and shown in FIG. 1 as being a separate and distinct component of the system 100, in some implementations, the control system 110 and/or the memory device 114 are integrated in the user device 170 and/or the respiratory device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system for analyzing data associated with a user's use of the respiratory therapy system 120, according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, the user device 170, and the blood pressure device 182 and/or activity tracker 190. As yet another example, a third alternative system includes the control system 110, the memory device 114, the respiratory therapy system 120, at least one of the one or more sensors 130, activity tracker 190 and the user device 170. As a further example, a fourth alternative system includes the control system 110, the memory device 114, the respiratory therapy system 120, at least one of the one or more sensors 130, the user device 170, and the blood pressure device 182 and/or activity tracker 190. Thus, various systems can be formed using any portion or portions of the components shown and described herein and/or in combination with one or more other components.

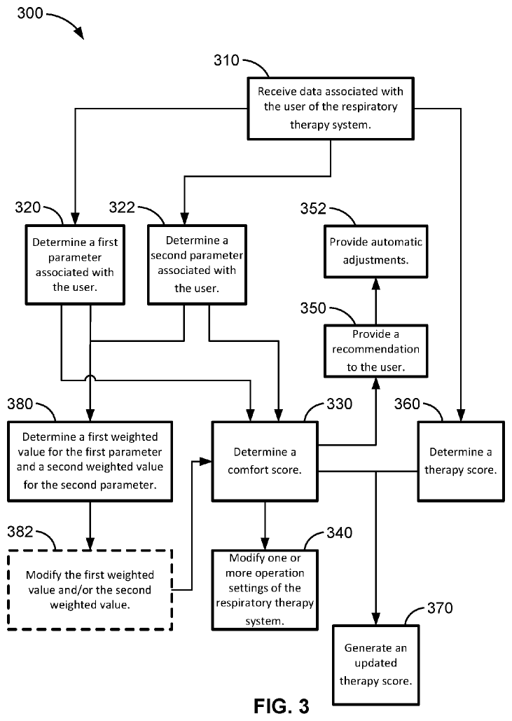

Referring to FIG. 3, a method 300 for estimating a subjective comfort level of a user of a respiratory therapy system is disclosed. The initial estimation of subjective comfort level may be based on objective data, and the estimation may be subsequently updated based on additional objective data and/or subjective data. A system implementing the method 300 can estimate the likely subjective comfort level of a user. For example, in some implementations, the system is a fully personalized system that can, by analyzing objective data, approximate what is actually experienced by the user (e.g., the user 210 in FIG. 2), and optionally the user's bed partner (e.g., the bed partner 220 in FIG. 2). Optionally, the system may also seek subjective data (such as via a question or questionnaire (e.g., based on the Epworth Sleepiness Scale), or other provided response to a stimulus or prompt) to add to and/or confirm the objective estimation. Such subjective data (e.g., subjective feedback) may be obtained from the user 210 and/or a third party, such as the bed partner 220 or a caregiver of the user 210. In other implementations, the system may not use or require subjective feedback from the user (and optionally bed partner or caregiver, etc.), and instead directly or indirectly effect a change in order to seek to increase the estimated comfort score (e.g., by automatically adapting one or more parameters of the system (such as a pressure setting of the respiratory device 122, a room environment setting, etc.), or provide such recommendations to a health care provider (HCP)/home medical equipment provider (HME) to make certain changes—e.g., to prescribe a different mask type or size, or different pressure settings/profile for the respiratory device 122, etc.).

In contrast to conventional sleep scores or therapy scores, a comfort score as described herein may be determined that is representative of the actual subjective comfort level of the user. For example, a therapy score for the user may be very good (i.e. indicating that the user received a good level of therapy), but the user may have been awake for some/most of the night, or only have used therapy for the required usage time, but less than the required sleep time. The comfort score described herein can include analysis of sleep, types of leak, any impact by or on a bed partner, room environment parameters, and data from previous therapy/sleep sessions and, as such, more closely represents the user's perception of their comfort—and thus be better able to affect changes to increase this comfort level either during the therapy/sleep session, and/or for future therapy/sleep sessions. The comfort score can be incorporated with a sleep score, a therapy score, or a combined sleep-therapy score, to provide a more accurate representation of the user's experience during a sleep or therapy session.

At step 310, data associated with the user of the respiratory therapy system is received. The received data may be associated with a sleep session and/or a therapy session, where the respiratory therapy system is used. The respiratory therapy system can include one or more components of the respiratory therapy system 120 (FIG. 1), and/or a mandibular repositioning device configured to adjust a position of a mandible of the user. The data may be received from the respiratory therapy system, one or more sensors (e.g., the sensors 130 of the system 100), a user device (e.g., the user device 170 of the system 100), a blood pressure device (e.g., the blood pressure device 182 of the system 100), an activity tracker (the activity tracker 190 of the system 100), or any combination thereof.

The data received at step 310 includes acoustic data, pressure data, flow rate data, moisture data, motion data (e.g., including user movement data), physiological data, user input data, or any combination thereof. The acoustic data can include noise related to the user (e.g., respiration-related noises, etc.), noise related to a user's environment (e.g., noise from traffic, noise from a TV, etc.), or both. The pressure data and flow rate data can be derived from the respiratory therapy system 120, and the pressure sensor 132 and flow sensor 134 comprised therein, and be indicative of the airflow properties of the pressurized air generated by the respiratory therapy system 120 and the influence of the user's respiration on that airflow. The moisture data can include a humidification level associated with a humidifier of the respiratory therapy system. Additionally or alternatively, the moisture data can include a presence and/or an amount of liquid in the conduit and/or the user interface (e.g., rainout). The motion data can be generated by a motion sensor 138 (such as a radar or sonar sensor described herein, an accelerometer worn on the body, etc.), and be indicative of the movement of the user 210 or a bed partner 220, etc. The physiological data can include breath alcohol data, blood alcohol data, blood pressure data, blood glucose data, blood oxygen data (e.g., SPO₂ level data), airway congestion data, airway occlusion data, body temperature data, heart rate data, respiration data, sleep stage data, CO₂ level data, or any combination thereof. The user input data can include information indicative of a subjective comfort level, a subjective stress level of the user, a subjective fatigue level of the user, a subjective health status of the user, a recent life event experienced by the user, objective health data associated with the user, or any combination thereof.

In some implementations, congestion (ranging from acute onset of congestion, chronic congestion, and conditions such as allergic rhinitis) can be detected via a microphone detecting the sound of sniffing, or via a sensor detecting a nasal drip, facial pain or pressure, or inferring from a change from nasal to mouth breathing when using a full face mask. Additionally or alternatively, in some implementations, lung compliance can be tracked based on processing on the respiratory device signals, such as to estimate pulmonary compliance—the ability of the lung to expand, stretch, distend in terms of static and dynamic components.

In some implementations, the objective health data associated with the user includes demographic and/or medical information of the user, such as an age of the user, a gender of the user, a race of the user, a geographic location of the user, a relationship status of the user, an employment status of the user, an educational status of the user, a socioeconomic status of the user, one or more medical conditions associated with the user, medication usage by the user, or any combination thereof.

Based at least in part on a first portion of the data received at step 310, at least one parameter associated with the user is determined. For example, at step 320, a first parameter associated with the user is determined. At step 322, a second parameter associated with the user is determined. The at least one parameter can include a leak type (e.g., mouth leak, unintentional user interface leak, intentional user interface leak, and/or conduit leak), a leak amount (e.g., total and/or for each leak type), a duration of leak (e.g., total and/or for each leak type), a duration of use, a type of events, a number of the type of events, a body position, a change in body position, a number of changes in body position, a motion change, a number of motion changes, a duration of residual snore, a residual score (e.g., a residual AHI score and/or a residual snore score), a severity of residual snore, a number of turnovers, a number of arousals (e.g., awakenings), a change in sleep architecture, a restless measure (e.g., a number of changes in body position, a number of times the user gets in/out of bed, a time it takes the user to fall asleep, a percentage of time during the sleep session where the user is awake, etc.), a number of pressure changes, a heart rate, a change in heart rate, a number of changes in heart rate, a heart rate variability, a respiration rate, a change in respiration rate, a number of changes in respiration rate, a respiratory rate variability, a pain location, a pain severity, a humidification efficacy level, or other physiological parameters indicative of physiological properties associated with the user, or any combination thereof. In relation to a restless measure, restlessness is a useful indicator of discomfort. Such discomfort may be caused by a user's sleeping environment, therapy and/or therapy equipment, or medical conditions (which may be co-morbidities) such as PLMD, COPD, etc. PLMD can disturb a user's sleep, therapy, or both, can also negatively a user's comfort score. PLMD may be detected by means such as described in US2018/0353138 A1, which is hereby incorporated by reference herein in its entirety. COPD typically negatively impacts a user's respiratory function and thus can disturb a user's sleep, therapy, or both, as well as negatively a user's comfort score. Other conditions (co-morbidities) such as hypertension, diabetes, insomnia, hyperarousal, etc. may also affect a user's comfort. Events or exacerbations related to those conditions may be detected by suitable sensors (e.g., a blood pressure monitor may detect hypertension, a glucose monitor detect blood glucose levels related to diabetes, a sleep monitor (e.g., sleep tracker based on radar or sonar technology as described herein) may detect insomnia, hyperarousal, etc.) and/or reported by the user, caregiver or other third party.

For example, body position can be related to comfort. Some users with positional apnea may be more comfortable sleeping on their side for most of the night as their pressure can be lower. Conversely, if a user has a history of back pain, they may have increased overall comfort if they can sleep on their back for more of the night, with a higher average pressure.

In some implementations, the type of events includes snoring, apneas, central apneas, obstructive apneas, mixed apneas, hypopneas, residual apnea events, a cough, a restless leg, a sleeping disorder, choking, an increased heart rate, labored breathing, an asthma attack, an epileptic episode, a seizure, increased blood pressure, or any combination thereof. Therefore, the system is capable of capturing, aggregating, and processing biometric and behavioral data from multiple data sources for the patient (user) and optionally for the user's bed partner.

At step 330, a comfort score is determined, based at least in part on the determined at least one parameter (e.g., the first parameter received at step 320 and/or the second parameter received at step 322). The comfort score determined at step 330 may be indicative of the subjective comfort level of the user of the respiratory therapy system during at least a portion of the therapy session. In some implementations, at step 380, a first weighted value for the first parameter and a second weighted value for the second parameter are determined. In some implementations, the initial weighting (e.g., for determining the first weighted value and the second weight value) is a default weighting learned and/or estimated from a population of users and/or non-users (e.g. of similar demographic). For example, an initial baseline set of weighted values (e.g., the base weight values 106 of the system 100) may be pre-learned, and/or updated based on accessing a cloud service to connect to the most recent learned initial weight values for the comfort parameters. Additionally or alternatively, in some implementations, the initial weighting is calculated based on a lookup table (e.g., Table 2). The population of users and/or non-users may be selected based on the user's own demographic information, and/or personal preferences regarding sleep and/or therapy comfort, the identity and/or severity of the user's sleep-/respiratory-related disorder, the user's therapy equipment and/or therapy prescription, or any combination thereof.

For example, some objective health data may affect the user's subjective comfort level to a greater extent than other objective health data, thus may be assigned a higher initial weighting. For example, having chronic pain (e.g., an initial weight of 8 on a scale of 0-10) may have a higher initial weighting than having dry skin (e.g., an initial weight of 3 on a scale of 0-10), because chronic pain affects the user's comfort level during the therapy session or sleep session more than dry skin.

The first weighted value and/or the second weighted value may be modified, at step 382, based at least in part on the data associated with the user of the respiratory therapy system received at step 310. The comfort score is then determined, at step 330, based at least in part on the modified first weighted value and the modified second weighted value from step 382.

An initial training sequence can be performed by collecting objective data from one or more sensors (such as on or connected with the respiratory therapy device, such as a PAP device) related to the system performance and/or user biometrics and/or from the room environment and/or user demographic details—and gathering contemporaneous data from the user by means of questions/questionnaire, response to prompts or other stimuli, and/or PREMS (patient-reported experience measures), etc. These data may then be combined and used to train an AI system (such as to allow it to learn) in order to create a baseline system with initial parameters. This can then be refined by supervised or unsupervised learning, such as to improve the estimation of a likely subjective comfort score.

In some implementations, the weighting may be modified based at least in part on a parameter change associated with a sleep stage and/or a motion. Additionally or alternatively, in some implementations, a weighted value is assigned initially to each parameter. In some such implementations, the weighted value is determined using an initial weight for each parameter from a lookup table (e.g., Table 2). In some other such implementations, the weighted value for each parameter is determined based on pre-learned data associated with the user of the system or other users, such as a population of users (e.g., categorized based on demographic data and/or health data). In some implementations, the weighed value is updated based on accessing a cloud service to connect to the most recent learned initial weight values for the comfort parameters. Then, the parameters that have the greatest impact on the user's comfort level (e.g., in terms of sleep quality, therapy efficacy, and/or therapy duration) are learned by an algorithm.

The comfort score may be reported, acted upon, and/or combined with a sleep/therapy score by normalizing the score to the user for which it is determined. For example, a comfort score for a user may be interpreted (e.g., normalized) based on the user's demographics, medical history, respiratory device 122 and/or respiratory device settings. For example, the comfort score may be normalized for a user based on the type of the user's respiratory therapy system, which system may be selected from a continuous positive airway pressure (CPAP) system, an automatic positive airway pressure (APAP) system, a bi-level positive airway pressure (BPAP) system, a variable positive airway pressure (VPAP) system, and a ventilator. In an example, a relatively low comfort score may be expected for a relatively young user, and thus the score may be normalised and interpreted as “good”, “acceptable”, or similar. In contrast, the same comfort score may be unexpected for a relatively old user, and thus the score may be normalised (or not) and interpreted as “bad”, “unacceptable (requires intervention)”, or similar. For example, the normalized comfort score may be outputted with (e.g., separately, or as part of) a therapy score and/or sleep score that is determined based at least in part on the received data associated with the user of the respiratory therapy system. Further, a therapy score and/or sleep score may be determined based at least in part on (i) the received data associated with the user of the respiratory therapy system, and (ii) the normalized comfort score.

In other examples, determination of the comfort score may be adjusted/updated over time as the system learns what parameter, or combination of parameters, influence a user's comfort. This may be achieved by, for example, collecting data on subjective comfort level from a user after a night's sleep and/or therapy and correlating with objective data/parameters collected (detected) during the corresponding sleep and/or therapy session. In this way, objectively determined parameter(s) can be correlated with an individual user's subjective comfort experience. For example, an objectively determined parameter such as dry mouth (e.g., caused by mouth breathing during respiratory therapy via a nasal mask) may cause discomfort for one user but may not for another user due to different subjective experiences of each user. In related examples, the objective data/parameters collected during a sleep and/or therapy session can be used by the system to select targeted questions/questionnaire for the user, or prompts for PREMS (patient-reported experience measures). Using the previous example, if dry mouth caused by mouth breathing by a nasal mask user is objectively determined, subjective data sought from the user may be related to dry mouth, at least.

In additional or alternative examples, the data related to one or more objectively determined parameters may be collected overtime, e.g., over a plurality of sleep and/or therapy sessions, and baseline levels and/or threshold levels for each parameter determined. This may be achieved by, for example, comparing an objectively determined parameter determined for one or more respiratory therapy sessions with the same parameter determined for one or more sleep sessions (or portions of sleep sessions) when the respiratory therapy system was not used. Additional or alternatively, subjective feedback for those sessions can be correlated with the objectively determined parameters. Additional or alternatively, objective data from different sources (e.g., sensors) may be correlated to help identify incidences and causes of discomfort for a user. For example, objectively determined dry mouth/mouth breathing may be correlated with objectively determined room and/or airflow humidity levels, objectively determined mask type, etc. This correlation can then inform the recommendation at step 350 and/or adjustments at step 352.

In some implementations, the system may be configured to generate messages/output to the user (and/or their bed partner) concerning comfort improvement, such as sleep advice as described in WO 2015/006364 and U.S. Pat. No. 10,376,670, each of which is hereby incorporated by reference herein in its entirety. For example, as the system builds an understanding of the respiratory therapy device (e.g., PAP device) usage, sleep patterns, biometrics patterns of the user, such as from sleep related analysis of sensor signals and questionnaires, the system may deliver customized personal advice to help improve the user's sleep and/or comfort through the utilization of an “advice engine.” In some such implementations, diagnostic capacities can be included in the advice engine to help identify other sleep issues, which can connect the user to other products such as for treatment of sleep related health issues (e.g., changes to PAP settings, changes to room environment, changes to medication, etc.). The advice, which is generated by one or more processors of the system, can be designed to inform the user of the benefits of good sleep habits, best environmental conditions for sleep, and/or daily activities that help sleep. It delivers credible and insightful information so as to assist the user's sleep, and keep the user engaged with the overall system. The system may implement a learning classifier, such as using Bayesian methods and/or a decision tree, in order to tailor advice to the individual patterns of the user, a local population, or a global population of system users. Further, in some implementations, the user can be prompted to respond to electronic queries embedded in task/advice nuggets received. The user responses can guide/trace a path through the contents of the decision tree. For example, in some implementations, each parameter may be ranked, and the parameters with greatest impact on the user's comfort level receive a higher weighting.

At step 340, one or more operation settings of the respiratory therapy system is modified, based at least in part on the comfort score determined at step 330. In some implementations, the modifying the one or more operation settings of the respiratory therapy system at step 340 includes adjusting pressure settings of a respiratory therapy device of the respiratory therapy system, adjusting humidification settings of a humidifier of the respiratory therapy system, or both. For example, in some implementations, adjusting pressure settings of the respiratory therapy device of the respiratory therapy system includes adjusting the blower motor in the respiratory therapy device.

At step 350, a recommendation is provided to the user, based at least in part on the comfort score determined at step 330. The recommendation may be provided to the user via a display device of the respiratory therapy system, a user device (e.g., audio and/or visual instruction via the user's smart phone), a speaker (e.g., audio instruction via a smart speaker), or any combination thereof. Additionally or alternatively, in some implementations, the recommendation may be (firstly, concurrently, subsequently, or only) provided to a caregiver (e.g., physician), HME, etc., who in turn conveys the recommendation to the user and/or takes an action (such as contacting the user or the user's caregiver, supplying a new user interface, prescribing a new therapy, prescribing an adjusted therapy, etc.).

Additionally or alternatively, in some implementations, the recommendation may be to investigate (e.g., consult physician/caregiver, triage, troubleshoot, etc.), begin, or modify therapy in relation to one or more medical conditions (e.g., co-morbidities) of the user, because these conditions may be, for example, inadequately treated (such as receiving no dedicated treatment). Such one or more medical conditions include PLMD, COPD, diabetes, hypertension, insomnia, hyperarousal, etc. and, as described above, may impact the user's comfort and be reflected in the comfort score. Additionally or alternatively, in some implementations, the recommendation may include a mapping of one or more parameters (on which the comfort score is based) to a comfort score or a plurality of comfort scores, such as plurality of comfort scores determined periodically over a sleep and/or therapy session. Such parameters can include one or more of arousals, blood oxygen (e.g., SpO₂) fluctuations, apnea events, leak events, etc. In this way, the user, caregiver or other third party may better understand the cause of a user's discomfort and help determine ways in which to improve the user's comfort.

In some implementations, the recommendation provided at step 350 includes (i) adjusting pressure settings of a respiratory therapy device; (ii) adjusting humidification settings of a humidifier coupled to the respiratory therapy device; (iii) recommending a mask type for the respiratory therapy system, (iv) recommending a sleep position for the user, (v) recommending a chin strap for the user; (vi) recommending a nasal cradle cushion for the user; or (vii) any combination thereof.

Further additionally or alternatively, at step 352, automatic adjustments may be made based at least in part on the comfort score determined at step 330 and/or the recommendation provided at step 350. For example, in a smart home, room temperature may be automatically reduced based at least in part on the comfort score determined at step 330. As another example, room humidity may be automatically increased based at least in part on the comfort score determined at step 330. Thus, the determined comfort score(s) may be used as part of a process or system to automatically adjust therapy settings including: (i) automatically adjusting pressure settings of a respiratory therapy device, the pressure settings being associated with pressurized air supplied to the airway of the user, (ii) automatically adjusting humidification settings of a humidifier coupled to the respiratory therapy device, the humidifier being configured to introduce moisture to the pressurized air supplied to the airway of the user, or (iii) both (i) and (ii). Additionally, or alternatively, the determined comfort score(s) may be used as part of a process or system to automatically adjust therapy in relation to one or more medical conditions (e.g., co-morbidities) of the user. Such one or more medical conditions include PLMD, COPD, diabetes, hypertension, insomnia, hyperarousal, etc. and, as described above, may impact the user's comfort and be reflected in the comfort score.

The comfort score described herein can be used to help determine adjustments (recommended, manually or automatically applied) to settings of the respiratory therapy system 120, e.g., flow and pressure settings of the respiratory device 122, and/or inform auto-adjustment settings (e.g., Autoset™ feature in ResMed respiratory therapy systems) or other comfort-related settings of the respiratory therapy system such as Expository Pressure Relief (EPR), which feature maintains the airflow pressure for the user during inhalation and reduces the pressure during exhalation making it easier and more comfortable for the user when breathing out. The comfort score can be used to determine such setting adjustments in real-time and/or pre-/post-therapy, to dynamically adjust settings based on any changes in the user's comfort score, which may occur during a sleep/therapy session or between sleep/therapy sessions. In this way, a user's therapy comfort is monitored, which may change during a sleep/therapy session or across multiple sleep/therapy sessions, and settings adjusted as necessary to maintain or improve the user's comfort. Therapy setting may be adjusted periodically (e.g., repeatedly every number of seconds, minutes, or hours) throughout the therapy session based on a comfort score determined periodically throughout the therapy session.

In some implementations, a replacement user interface (e.g., replacement mask) is recommended at step 350. Additionally or alternatively, in some implementations, the replacement user interface is automatically processed (e.g., automatically ordered from a HME) and shipped to the user. This could be to replace a worn mask with a like for like replacement, or with a different type of mask that has been selected to further increase comfort (e.g., moving from a full face to nasal (or pillow) mask, or vice versa). In some implementations, the method also provides for recommending and/or automatically processing a replacement of a conduit (e.g., to move from an unheated to a heated conduit, to a different diameter or length of conduit, to a replacement waterless humidification module or element, or to a part of a user interface, such as a replacement silicon or foam element).

The comfort score can be used to help determine users that are likely to abandon, or not correctly adhere to, therapy. A comfort score can be determined for a user and compared to a predetermined threshold. If the user's comfort score is below the threshold (or above, depending on the nature of the threshold), or a number of comfort scores calculated over a predetermined number of sleep/therapy sessions is below (or above) a threshold, an intervention by the user, a caregiver, or another party may be recommended. Thus, the comfort score can be used to feed into a risk management tool or system for monitoring a user in terms of risk such as health risk, compliance risk, etc. As such, the comfort score can be used to help predict a likelihood that the user will (i) not adhere to a therapy plan (e.g., a therapy plan prescribed by a physician), (ii) abandon the therapy plan, or (iii) both (i) and (ii), based at least in part on the determined comfort score or a trend of determined comfort scores. For example, a comfort score that exceeds (or falls below) a predetermined threshold, or a (negative) trend of comfort scores from a number of therapy sessions, may indicate that a user is likely to abandon/not adhere to therapy since the user is unacceptably uncomfortable. In other examples, the comfort score can feed into a coaching tool or system, with coaching instructions or material informed by (e.g., selected based on) the comfort score (e.g., plurality of historical comfort scores) of the user. Based on the comfort score, the coaching system may instruct the user in relation to their sleep hygiene, their sleep environment, their therapy equipment, etc., or any combination thereof.

In some implementations, at step 360, a therapy score and/or a sleep score is determined, based at least in part on the data associated with the user of the respiratory therapy system received at step 310. At step 370, an updated therapy score and/or sleep score may be generated based at least in part on the comfort score determined at step 330 and the therapy score determined at step 360. In some implementations, an initial sleep score represents an analysis of the sleep patterns throughout the sleep session, but not how refreshed the user feels after awakening. The comfort score can better represent how the user feels immediately after they wake, and sometime after they wake (such as minutes or hours later), which could have differing levels of comfort. In order to provide a more realistic sleep score, the comfort score could be used to adjust the sleep score up or down as needed. For example, if an initial sleep score of 92/100 is calculated for a user, but the comfort score is low for that sleep session, the sleep score may be adjusted based on the comfort score (such as by multiplying the initial sleep score by a factor calculated from comfort score).

In some implementations, the determining the comfort score at step 330 includes comparing the determined at least one parameter with a plurality of historical values for the same parameter. The plurality of historical values for the same parameter is associated with a plurality of corresponding historical subjective comfort levels. For example, in some such implementations, the received data includes objective data associated with the respiratory therapy system during the therapy session.

In some implementations, the plurality of historical values and the plurality of corresponding historical subjective comfort levels are obtained from stored data associated with (i) the user of the respiratory therapy system, (ii) one or more other users of one or more other respiratory therapy systems, or (iii) both (i) and (ii). For example, in some such implementations, the plurality of corresponding historical subjective comfort levels is received from (i) the user of the respiratory therapy system, (ii) the one or more other users of the one or more other respiratory therapy systems, or (iii) both (i) and (ii), such as via user input.

In some implementations, the plurality of historical values is associated with the plurality of corresponding historical subjective comfort levels by first receiving historical objective data and historical subjective comfort levels associated with a plurality of historical sleep sessions, a plurality of historical therapy sessions, or both. In some implementations, the historical objective data is received from at least two different sensors and then correlated with the historical subjective comfort levels. The received historical objective data is processed to extract a plurality of historical values. A historical subjective comfort level of the plurality of historical subjective comfort levels is correlated with one or more historical values of the plurality of historical values. In some such implementations, the plurality of historical sleep sessions is associated with not using a respiratory therapy system, and the plurality of historical therapy sessions is associated with using a respiratory therapy system.

In some such implementations, the method 300 further includes determining a baseline level for a parameter of the at least one parameter, based at least in part on comparing (i) the corresponding historical parameter extracted from objective data received during one or more historical sleep sessions to (ii) the corresponding historical parameter extracted from objective data received during one or more historical therapy sessions.

Generally, the method 300 can be implemented using a system having a control system with one or more processors, and a memory storing machine readable instructions. The controls system can be coupled to the memory; the method 300 can be implemented when the machine readable instructions are executed by at least one of the processors of the control system. The method 300 can also be implemented using a computer program product (such as a non-transitory computer readable medium) comprising instructions that when executed by a computer, cause the computer to carry out the steps of the method 300.

While the system 100 and the method 300 have been described herein with reference to a single user, more generally, the system 100 and the method 300 can be used with a plurality of users simultaneously (e.g., two users, five users, 10 users, 20 users, etc.). For example, the system 100 and the method 300 can be used in a cloud monitoring setting.

Additionally, or alternatively, in some implementations, the system 100 and/or the method 300 can be used to monitor one or more users while using one or more respiratory systems (e.g., a respiratory therapy system described herein). For example, in some such implementations, a notification associated with the subjective comfort level and/or the comfort score associated with the one or more users can be sent to a monitoring device or personnel. The subjective comfort level and/or the comfort score can be determined using one or more steps of the method 300. Additionally, or alternatively, in some implementations, the subjective comfort level and/or the comfort score are simply being recorded.

### Example Implementations

As previously described with reference to FIG. 1, the comfort score is determined by a comfort score module 102, and is a function of the comfort parameters 104 and base weight values 106. An example implementation of base weight values 106 is shown with reference to Table 1, which lists base weight values (from a scale from 0 to 10) and comfortable thresholds for heart rate and heart rate variability of the user.

TABLE 1
Example Base Weight Values

| | | | Base weight value | | | | Base weight value | | | |
|---|---|---|---|---|---|---|---|---|---|---|
| | Comfort | | Short term time | | | | Long term time | | | |
| | Thresholds | | | Stable- | Stable- | | | Stable- | Stable- | |
| Parameter | Low | High | + | good | bad | − | + | good | bad | − |
| Heart rate (beats | 40 | 100 | 4 | 6 | 7 | 6 | 5 | 6 | 7 | 8 |
| per minute) | | | | | | | | | | |
| Heart rate | 50 | 70 | 5 | 7 | 8 | 7 | 6 | 7 | 8 | 8 |
| variability | | | | | | | | | | |
| (milliseconds) | | | | | | | | | | |

There are many ways of calculating heart rate variability (HRV) from inter-beat intervals—where these “intervals” are initially estimated between detected or estimated (e.g., where some form of interpolation may have been applied to combat artifact such as noise) fiducial points, such as from cardiac “RR” interbeats (RRI) or from optical sensors based on PPG or otherwise. For example, the RRI in ms could be used directly. Alternatively, in some implementations, further processing may be applied in the time domain to obtain the mean NN (average RR intervals over given time—where “N” indicates normal, where unexpected beats have been removed), the SDNN (average RR intervals over time), the RMSSD (standard deviation of normal RR intervals). Frequency domain based metrics could include low frequency (LF—often 0.03-0.15 Hz is used here), high frequency (HF—often 0.15-0.40 Hz is used here) and ratios of LF:HF.

A parameter value is considered comfortable if it is between the low and the high thresholds. In this implementation, if the user's heart rate is between 40 beats per minute (bpm) to 100 bpm, these are considered comfortable values for the heart rate. Similarly, if the user's heart rate variability (HRV) is between 50 to 70 ms, these are considered comfortable values for the HRV. The comfort thresholds can be initially set based on the user profile associated with the user (e.g., demographic information, medical information, age, and gender), and/or additional data collected on the user (e.g., data received at step 310 of the method 300). In some implementations, there is only a low threshold, or only a high threshold. The initially set thresholds can be adjusted based on changes in the user profile.

In some implementations, the heart rate data may be further analyzed. For example, absolute heart rate at night may be processed. As another example, a normalized heart rate may be processed based on the full night or values from previous nights. The variation from the normalized values may better remove user-to-user variability, and the residue can be used for trend analysis (e.g., looking for increasing, the same, or decreasing trends over a time period) from wake to sleep, and between different sleep stages. In some implementations, if the person is paced, a correction factor may be applied to RR and HRV estimates.

In some implementations, the HRV may be estimated using spectral measures, such as determining LF/HF ratios. If LF/HF ratios are used, we can compare to mean, trimmed mean, or median, rather than RR interval in ms as discussed herein. In some implementations, the HRV may be analyzed at shorter timescales, which would provide subtler variations.

In some implementations, HRV may be higher during NREM than REM, with both NREM and REM higher than wake. Therefore, in a “normal” sleep cycle variation, where we expect more deep sleep early in the night, and more REM towards the end of the night, the system would expect greater HRV during estimated deep sleep N3 (SWS). Maximal HRV is likely to coincide with deepest sleep (sustained SWS) which, dependent on time to bed, may be in the early hours (1-3 am). In addition, HRV usually increases at night during sleep, as there is a higher vagal (parasympathetic) balance versus sympathetic.

In some implementations, the determination of comfort score may be personalized by comparing a daytime HRV estimate (e.g., from a smartwatch) to a night-time HRV estimate. The system checks whether the expected increase is present to indicate comfort, or not, and estimates why. Daytime HRV (either via RR interval analysis, or spectral LF/HF analysis) along with sleep can have an overlaid circadian rhythm, so time of day/night is used by the system, overlaid with a circadian model of sleepiness. A correction factor may be applied by the system where a chronic condition exists (particularly a cardiac condition such as post MI), where the expected HRV increases during sleep are affected by the underlying condition (e.g., a user with a managed condition may still be comfortable, or uncomfortable) at lower levels of HRV difference; and then this correction is made to better personalize the comfort score to the user

For example, where a user is very active, the comfort high threshold for the example heart rate of Table 1 can be decreased below 100 bpm. Conversely, a user who may have a new health issue, such as a broken hip due to a fall, would require an upward shift in the high threshold for the heart rate. As another example, heart rate variability is usually higher in younger people, in those sleeping better, and in those with lower stress levels. So having access to the age of the user, their underlying health, and any stressors (chronic or acute), as well as estimated sleep quality can aid in estimating the subjective comfort level of the user (whereby we have a shorter term dis-improvement in heart rate variability from the expected level for that user).

In some implementations, the base weight values may be determined and/or modified using long short-term memory (an artificial recurrent neural network (RNN) architecture) that learns patterns over time along with recurrent neural networks.

Base weight values for a short term time (e.g., 1, 2, or 3 days), and a long term time (e.g., 10 days, 1 month, or 6 months) are listed in Table 1. In some implementations, these are further categorized responsive to the trends seen for the comfort parameters over time. For example, in some such implementations, a “+” indicates a positive or good trend, “−” indicates a negative or bad trend. The category “stable-good” denotes that the trend is stable and within the comfortable thresholds. The category “stable-bad” denotes that the trend is stable, but the parameter or some combination of the parameters is outside the comfortable thresholds.

A positive or good trend indication generally indicates that the values associated with a parameter, during a given time period, are trending in a desired direction. Conversely, a negative trend indication generally indicates that the values associated with a parameter, during a given time period, are trending away from the desired direction. For example, for the parameter of heart rate variability, the trend indication takes the sign of the slope, which maps the values associated with the parameter over time (e.g., during a sleep session or several sleep sessions). For example, a positive slope indicates a positive trend indicator, and a negative slope denotes a negative trend indicator. For some other comfort parameters, the trend indication may take the opposite sign of the slope. For example, if the comfort parameter is blood pressure, the relationship is reversed. That is, generally, a lowering of blood pressure would denote a positive trend indicator, and an increase in blood pressure would denote a negative trend indicator.

The trend indication can aid in (1) determining whether it is a temporary comfort issue (e.g., requiring small adjustments such as pressure, humidity), or a chronic issue (e.g., requiring new prescription or other medical attention), and (2) whether the user's baseline needs to be adjusted (e.g., a user may have a small HRV, which ordinarily indicates less comfort, however the user might be quite comfortable because their baseline HRV is consistently small over the long term time).

Other comfort parameters can be treated similarly as described for heart rate and heart rate variability. In some implementations, a plurality of data points associated with comfort parameters, such as provided by one or more of sensors 130, can be used to determine a trend indication for each of the comfort parameters. In some implementations, the data set for each of the comfort parameters is normalized. This normalization can simplify the analysis and manipulations, for example, by allowing selection of a meaningful and single upper and single lower slope thresholds, and having base weight values of similar magnitude for all the parameters.

Other categories of additional base weight values and for determining the additional base weight values are contemplated. For example, where a trend is positive but entirely outside of the comfortable thresholds, a category of “positive-bad” can be used. For a trend that is negative and entirely outside the comfortable thresholds, a category of “negative-bad” can be used.

Although only describing a long time period and a short time period, other additional time periods are contemplated. For example, a very-long time period can be greater than the long time period. Although the total specified days for the long term time included day 1 through 30, longer periods of time can be used. For example, data used for determining comfort parameters can be collected for more than 30 days, more than three months, more than six months, more than a year or for more than several years (e.g., 2, 3, 4, 5 or more years).

Optionally, in some implementations, the trend indication for a first parameter is a stable-good trend indication responsive to determining that (i) the slope of the fitted line is within a range of slope values, and (ii) an average of at least two values (e.g., a first value and a second value) of the first parameter is within a range of comfortable threshold values. In some implementations, at least one of the first value and the second value are within the range of comfortable threshold values for the trend indication to stable-good. In some implementations, the first value and the second value are within the range of the comfortable threshold values for the trend indication to be stable-good.

Optionally, in some implementations, the trend indication for a first one of the plurality of parameters is a stable-bad trend indication responsive to determining that (i) the slope of the fitted line is within a range of slope values, and (ii) an average of at least the first value and the second value is outside a range of comfortable threshold values. In some implementations, at least one of the first value and the second value are outside of the range of comfortable threshold values for the trend indication to be stable-bad. In some implementations, at least the first value and the second value are outside of the range of comfortable threshold values for the trend indication to be stable-bad.

In some implementations, the comfort score is determined as a sum of a first product and a second product. The first product is the product of the second value for the first parameter and the associated determined base weight value for the first parameter. The second product is the product of the second value for the second parameter and the associated determined base weight value for the second parameter. Equation 1 is a mathematical expression of this function.

CS=W₁P₁+W₂P₂ (1)

where CS is the comfort score; W₁ is the determined base weight for the first parameter P₁; and W₂ is the determined base weight for the second parameter P₂.

Equation 1 can be expanded to include additional parameters, and can be expressed as the sum of products over all the relevant parameters as shown in Equation 2.

CS=Σₖ₌₁ⁿWₖPₖ (2)

where n is the number of relevant parameters. In some implementations, a parameter may be determined to have no effect to the user's subjective comfort, therefore, for that parameter Pₘ, the product WₘPₘ=0.

Table 2 provides a neutral adjusted scale from 0 to 10 for selected example parameters, where 5 is feeling comfortable, greater than 5 is better than comfortable (e.g., a level of comfort greater than required for a comfortable night's sleep), and less than 5 is feeling worse than comfortable (e.g., a level of comfort less than required for a comfortable night's sleep). Table 2 illustrates sources, and initial weights of the example parameters. As disclosed herein, in some implementations, the initial weight for each parameter is looked up from Table 2. In some such implementations, the weighted value for each parameter is determined based on pre-learned data associated with the user of the system or other users (e.g., categorized based on demographic data and/or health data). For example, the initial weights for a first user may be different from the initial weights for a second user, due to their respective demographic data and/or health data.

TABLE 2
Example Parameters

| | | | Initial | | |
|---|---|---|---|---|---|
| Parameter | Description | Source | Weight | Scale | Type |
| heart rate | average beats per minute for a | Vitals; | 4 | 0-10 | Objective |
| | period of time (e.g., 1 hour) | sensor | | | |
| heart rate | variance in time between the | Vitals; | 7 | 0-10 | Objective |
| variability | heart beats (e.g., 60 ms) | sensor | | | |
| User interface | amount of leak per unit time | Sensor | 5 | 0-10 | Objective |
| leak | (e.g., liters per minute) for a | | | | |
| | period of time (e.g., 1 hour) | | | | |
| mouth leak | amount of mouth leak per unit | Sensor | 4 | 0-10 | Objective |
| | time (e.g., liter per minute) for a | | | | |
| | period of time (e.g., 1 hour) | | | | |
| pain (e.g. back | intensity of pain experienced for | Questionnaire; | 4 | 0-10 | Subjective |
| pain) | a period of time (e.g., 1 hour or 1 | sensor | | | |
| | sleep session) | | | | |
| body position | number of position changes from | Sensor | 3 | 0-10 | Objective |
| changes | prone, left/right, supine for a | | | | |
| | period of time (e.g. 1 hour) | | | | |
| motion changes | number of motion events of | Sensor | 4 | 0-10 | Objective |
| | intensity and duration above a | | | | |
| | threshold | | | | |
| residual AHI | number of apnea/hypopnea | Sensor | 4 | 0-10 | Objective |
| | events | | | | |
| self-reported | self-reported comfort for night - | Questionnaire; | 7 | 0-10 | Subjective |
| comfort | VAS (visual analog scale) or list | sensor | | | |
| | checkbox | | | | |
| Humidification | related to humidifier present, | Sensor | 2 | 0-10 | Objective |
| efficacy level | operation, water available, | | | | |
| | heated tube available, no rainout | | | | |
| respiration rate | average breaths per minute for a | Vitals; | 4 | 0-10 | Objective |
| | period of time (e.g., 1 hour) | sensor | | | |
| respiration rate | variance in time between the | Vitals; | 5 | 0-10 | Objective |
| variability | breaths (e.g., 60 ms) | sensor | | | |
| longitudinal data | prior session(s) data - thresholds | | 5 | 0-10 | |
| | adapted | | | | |

Various aspects of the present disclosure can be performed by a machine-learning algorithm, as readily understood by a person skilled in the art. In some examples, step 330 or step 360 of FIG. 3 can be performed by a supervised or unsupervised algorithm. For instance, the system may utilize more basic machine learning tools including (1) decision trees (“DT”), (2) Bayesian networks (“BN”), (3) artificial neural network (“ANN”), or (4) support vector machines (“SVM”). In other examples, deep learning algorithms or other more sophisticated machine learning algorithms, e.g., convolutional neural networks (“CNN”), recurrent neural networks (“RNN”), or capsule networks (“CapsNet”) may be used.

DT are classification graphs that match input data to questions asked at each consecutive step in a decision tree. The DT program moves down the “branches” of the tree based on the answers to the questions (e.g., First branch: Did the subjective data include certain input? yes or no. Branch two: Did the objective data include certain parameters? yes or no, etc.).

Bayesian networks (“BN”) are based on likelihood something is true based on given independent variables and are modeled based on probabilistic relationships. BN are based purely on probabilistic relationships that determine the likelihood of one variable based on another or others. For example, BN can model the relationships between objective data, subjective data, and any other information as contemplated by the present disclosure. Particularly, if a historical comfort level and particular parameters of the user's objective data are known, a BN can be used to compute a current comfort score. Thus, using an efficient BN algorithm, an inference can be made based on the input data.

Artificial neural networks (“ANN”) are computational models inspired by an animal's central nervous system. They map inputs to outputs through a network of nodes. However, unlike BN, in ANN the nodes do not necessarily represent any actual variable. Accordingly, ANN may have a hidden layer of nodes that are not represented by a known variable to an observer. ANNs are capable of pattern recognition. Their computing methods make it easier to understand a complex and unclear process that might go on during determining a comfort level based a variety of input data.

Support vector machines (“SVM”) came about from a framework utilizing of machine learning statistics and vector spaces (linear algebra concept that signifies the number of dimensions in linear space) equipped with some kind of limit-related structure. In some cases, they may determine a new coordinate system that easily separates inputs into two classifications. For example, a SVM could identify a line that separates two sets of points originating from different classifications of events or parameters.

Deep neural networks (DNN) have developed recently and are capable of modeling very complex relationships that have a lot of variation. Various architectures of DNN have been proposed to tackle the problems associated with algorithms such as ANN by many researchers during the last few decades. These types of DNN are CNN (Convolutional Neural Network), RBM (Restricted Boltzmann Machine), LSTM (Long Short Term Memory) etc. They are all based on the theory of ANN. They demonstrate a better performance by overcoming the back-propagation error diminishing problem associated with ANN.

Machine learning models require training data to identify the parameters of interest that they are designed to detect. For instance, various methods may be utilized to form the machine learning models, including applying randomly assigned initial weights for the network and applying gradient descent using back propagation for deep learning algorithms. In other examples, a neural network with one or two hidden layers can be used without training using this technique.

In some examples, the machine learning model can be trained using labeled data, or data that represents certain user input. In other examples, the data will only be labeled with the outcome and the various relevant data may be input to train the machine learning algorithm.

For instance, to determine whether particular comfort level fits the input data, various machine learning models may be utilized that input various data disclosed herein. In some examples, the input data will be labeled by having an expert in the field label the relevant observations according to the particular situation. Accordingly, the input to the machine learning algorithm for training data identifies various data as from health users from users with various sleep disorders.

One or more elements or aspects or steps, or any portion(s) thereof, from one or more of any of claims 1-47 below can be combined with one or more elements or aspects or steps, or any portion(s) thereof, from one or more of any of the other claims 1-47 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

What is claimed is:

1. A method for estimating a subjective comfort level of a user of a respiratory therapy system, the method comprising:
receiving data associated with the user of the respiratory therapy system during a therapy session;
determining a first parameter associated with the user based at least in part on a first portion of the received data; and
determining a comfort score based at least in part on a first weighted value for the determined first parameter and a second weighted value for a second parameter, the comfort score being indicative of the subjective comfort level of the user of the respiratory therapy system during at least a portion of the therapy session.

2. The method of claim 1, wherein the respiratory therapy system includes a respiratory therapy device configured to supply pressurized air to an airway of the user, a mandibular repositioning device configured to adjust a position of a mandible of the user, or both.

3. The method of claim 1, wherein the data associated with the user is received from the respiratory therapy system, a user device, a sensor, or any combination thereof.

4. (canceled)

5. The method of claim 3, wherein the sensor is coupled to the respiratory therapy system and/or the user device.

6. The method of claim 1, wherein the at least one parameter includes a leak type, a leak amount, a duration of leak, a duration of use, a type of event, a number of the type of events, a body position, a change in body position, a number of changes in body position, a motion change, a number of motion changes, a duration of residual snore, a residual score, a severity of residual snore, a number of turnovers, a number of arousals, a change in sleep architecture, a restless measure, a number of pressure changes, a heart rate, a change in heart rate, a number of changes in heart rate, a heart rate variability, a respiration rate, a change in respiration rate, a number of changes in respiration rate, a respiratory rate variability, a pain location, a pain severity, a humidification efficacy level, or any combination thereof.

7. The method of claim 6, wherein the residual score includes a residual AHI score, a residual snore score, or both, and wherein the leak type includes mouth leak, unintentional user interface leak, intentional user interface leak, conduit leak, or any combination thereof and wherein the type of events includes snoring, apneas, central apneas, obstructive apneas, mixed apneas, hypopneas, residual apnea events, a cough, a restless leg, a sleeping disorder, choking, an increased heart rate, labored breathing, an asthma attack, an epileptic episode, a seizure, increased blood pressure, or any combination thereof.

8-9. (canceled)

10. The method of claim 1, further comprising based at least in part on the determined comfort score, determining a modification to one or more operation settings of the respiratory therapy system.

11. (canceled)

12. The method of claim 1, further comprising based at least in part on the determined comfort score, providing a recommendation to the user, wherein the recommendation includes (i) adjusting pressure settings of a respiratory therapy device, the pressure settings being associated with pressurized air supplied to the airway of the user; (ii) adjusting humidification settings of a humidifier coupled to the respiratory therapy device, the humidifier being configured to introduce moisture to the pressurized air supplied to the airway of the user; (iii) recommending a mask type for the respiratory therapy system, (iv) recommending a sleep position for the user, (v) recommending a chin strap for the user; (vi) recommending a nasal cradle cushion for the user; or (vii) any combination thereof.

13. (canceled)

14. The method of claim 1, wherein the received data includes acoustic data, pressure data, flow rate data, moisture data, motion data, physiological data, user input data, or any combination thereof.

15. (canceled)

16. The method of claim 14, wherein the user input data includes information indicative of a subjective comfort level, a subjective stress level of the user, a subjective fatigue level of the user, a subjective health status of the user, a recent life event experienced by the user, objective health data associated with the user, or any combination thereof.

17. The method of claim 16, wherein the objective health data associated with the user includes demographic and/or medical information of the user, such as an age of the user, a gender of the user, a race of the user, a geographic location of the user, a relationship status of the user, an employment status of the user, an educational status of the user, a socioeconomic status of the user, one or more medical conditions associated with the user, medication usage by the user, or any combination thereof.

18-19. (canceled)

20. The method of claim 1, further comprising modifying the first weighted value and/or the second weighted value based at least in part on a second portion of the received data associated with the user of the respiratory therapy system, wherein the comfort score is determined based at least in part on the modified first weighted value and the modified second weighted value.

21. The method of claim 1, further comprising determining a therapy score and/or sleep score based at least in part on the received data associated with the user of the respiratory therapy system.

22. The method of claim 1, further comprising normalizing the comfort score based at least in part on a demographic parameter of the user, a medical history of the user, a type of the respiratory therapy system or component thereof, a setting of the respiratory therapy system, or any combination thereof.

23-27. (canceled)

28. The method of claim 1, wherein the determining the comfort score includes comparing the determined at least one parameter with a plurality of historical values for the same parameter associated with a plurality of corresponding historical subjective comfort levels.

29-31. (canceled)

32. The method of claim 28, wherein the plurality of historical values is associated with the plurality of corresponding historical subjective comfort levels by:
receiving historical objective data and historical subjective comfort levels associated with a plurality of historical sleep sessions, a plurality of historical therapy sessions, or both;
processing the received historical objective data to extract a plurality of historical values; and
correlating a historical subjective comfort level of the plurality of historical subjective comfort levels with one or more historical values of the plurality of historical values.

33-35. (canceled)

36. The method of claim 1, further comprising automatically adjusting a therapy setting based on the determined comfort score including (i) automatically adjusting pressure settings of a respiratory therapy device, the pressure settings being associated with pressurized air supplied to the airway of the user, (ii) automatically adjusting humidification settings of a humidifier coupled to the respiratory therapy device, the humidifier being configured to introduce moisture to the pressurized air supplied to the airway of the user, or (iii) both (i) and (ii).

37-39. (canceled)

40. The method of claim 1, further comprising predicting a likelihood that the user will (i) not adhere to a therapy plan, (ii) abandon the therapy plan, or (iii) both (i) and (ii), based at least in part on the determined comfort score or a trend of determined comfort scores.

41-43. (canceled)

44. A computer program product comprising instructions which, when executed by a computer, cause the computer to carry out the steps of:
receiving data associated with the user of the respiratory therapy system during a therapy session;
determining a first parameter associated with the user based at least in part on a first portion of the received data; and
determining a comfort score based at least in part on a first weighted value for the determined first parameter and a second weighted value for a second parameter, the comfort score being indicative of the subjective comfort level of the user of the respiratory therapy system during at least a portion of the therapy session.

45. (canceled)

46. A system comprising:
a control system including one or more processors; and
a memory having stored thereon machine readable instructions;
wherein the control system is coupled to the memory and is configured to implement a method for estimating a subjective comfort level of a user of a respiratory therapy system, the method comprising:
receiving data associated with the user of the respiratory therapy system during a therapy session;
determining a first parameter associated with the user based at least in part on a first portion of the received data; and
determining a comfort score based at least in part on a first weighted value for the determined first parameter and a second weighted value for a second parameter, the comfort score being indicative of the subjective comfort level of the user of the respiratory therapy system during at least a portion of the therapy session.

47. (canceled)
